(19) United States
(12) Patent Application Publication
(10) Pub. No.: US 2011/0218114 A1
(43) Pub. Date: Sep. 8, 2011

(54) NUCLEOTIDE TRIPHOSPHATE WITH AN ELECTROACTIVE LABEL CONJUGATED TO THE GAMMA PHOSPHATE

(76) Inventors: Heinz-Bernhard Kraatz, London (CA); Kagan Kerman, Toronto (CA); Haifeng Song, London (CA)

(21) Appl. No.: 12/680,342

(22) PCT Filed: Sep. 26, 2008
(86) PCT No.: PCT/CA2008/001690

§ 371 Date: Sep. 9, 2010

Related U.S. Application Data
(60) Provisional application No. 60/960,398, filed on Sep. 28, 2007.

(51) Int. Cl. C40B 30/00; C07H 19/20; C07H 23/00; G01N 33/50; G01N 27/327

(52) U.S. Cl. 506/7; 536/26.26; 205/777.5; 204/403.14

(57) ABSTRACT

A nucleotide triphosphate (NTP) participates in a phosphorylation reaction, wherein a phosphate group is transferred from the NTP to a substrate by a kinase. Provision in a kinase reaction of a NTP whose gamma phosphate is conjugated to an electroactive label results in the transfer of the gamma phosphate-electroactive label conjugate from the NTP to the substrate. The electroactive label is an organic moiety such as a quinone or a nitroheterocycle, or is a metallocene such as a ferrocene or a cobaltocene. Upon transfer of the gamma phosphate-electroactive label conjugate to an electrode-bound substrate by a kinase, the phosphorylation event is detected electrochemically by cyclic voltammetry. Phosphorylation can also be detected by mass spectrometry of a substrate carrying the electroactive label-conjugated gamma phosphate. NTP comprising the gamma phosphate-electroactive label conjugate is used in methods of detecting the presence of a kinase in a sample, screening candidate compounds that modulate kinase activity, and in methods of diagnosing a disease associated with a kinase.

### PRIORITY APPLICATION

This application claims priority from U.S. provisional application No. 60/960,398 filed Sep. 27, 2007.

### FIELD OF THE INVENTION

The present invention relates to a novel electroactive nucleotide triphosphate useful to monitor events associated with phosphorylation.

### BACKGROUND OF THE INVENTION

In the cellular communication network, many enzymes and receptors are switched “on” or “off', or in other terms, “phosphorylated” and “dephosphorylated”. During phosphorylation, a phosphoryl group from ATP is transferred to specific serine, threonine, or tyrosine residue of a protein. As a result of these modifications, the function or localization of the protein may change, which in some cases may lead to the formation of oncoproteins.¹

Abnormal protein phosphorylation is a cause of major diseases, including cancer, diabetes and chronic inflammatory diseases.² Analytical methods to quantify protein kinase activity are critical for understanding their role in the diagnosis and therapy of these diseases. Current methods for the detection of protein phosphorylation rely on radio-labeled. ATP,³ fluorescence-based methods,⁴ and fluorescence resonance energy transfer (FRET).⁵ Recently, biotin-conjugated ATP molecules have been exploited for the detection of phosphorylation reactions.⁶ However, additional modification of the peptides with an electro-active or optical label is necessary, which increases the cost and causes tedious and time-consuming handling procedures.

It would be desirable, thus, to develop an alternative method of monitoring or detecting events associated with phosphorylation which overcomes at least one of the disadvantages of the current detection methods.

### SUMMARY OF THE INVENTION

A novel electroactive nucleotide triphosphate has now been developed which is useful in an alternative method of monitoring and/or detecting events associated with phosphorylation, including phosphorylation itself.

Thus, in one aspect of the present invention, a nucleotide triphosphate conjugate comprising an electroactive labelled gamma phosphate group is provided.

In another aspect of the invention, a method of detecting the phosphorylation of a kinase substrate is provided comprising:
(a) immobilizing the substrate on an electrode surface;
(b) incubating the immobilized substrate with a kinase and a nucleotide triphosphate conjugate comprising an electroactive-labelled gamma phosphate under conditions which permit detection of phosphorylation activity; and
(c) detecting phosphorylation of the substrate.

In one aspect the phosphorylation is detected electrochemically. In another aspect the phosphorylation is detected by spectroscopy, including mass spectroscopy.

In another aspect of the invention, a method of detecting a kinase of interest in a sample is provided comprising:
(a) immobilizing a substrate specific for the kinase of interest on an: electrode surface;
(b) incubating the immobilized substrate with the sample and a nucleotide triphosphate conjugate comprising an electroactive labelled gamma phosphate under conditions which permit detection of phosphorylation activity; and
(c) detecting phosphorylation of the substrate,
   wherein phosphorylation of the substrate indicates the presence of the kinase in the sample.

In yet another aspect of the invention, a method of identifying a candidate kinase substrate is provided comprising:
(a) immobilizing the candidate kinase substrate on an electrode surface;
(b) incubating the immobilized substrate with a kinase-containing electrolyte and a nucleotide triphosphate conjugate comprising an electro-active labelled gamma phosphate under conditions which permit detection of phosphorylation activity; and
(c) detecting phosphorylation of the substrate, wherein phosphorylation of the candidate substrate indicates that said candidate is a substrate of the kinase.

In another aspect of the invention, a method of screening candidate compounds that modulate kinase activity is provided comprising:
(a) immobilizing a substrate of a kinase on an electrode surface;
(b) incubating the immobilized substrate with a kinase, a candidate compound and a nucleotide triphosphate comprising an electroactive-labelled gamma phosphate under conditions which permit detection of phosphorylation activity; and
(c) detecting a level of phosphorylation of the substrate, wherein a change in the phosphorylation level from a level of phosphorylation that is achieved in the absence of said compound indicates that said compound modulates the activity of the kinase.

In another aspect of the invention, a method of high-throughput screening a sample for the presence of protein kinases is provided comprising:
(a) providing a microelectrode array comprising a plurality of electrodes;
(b) immobilizing kinase substrates to each electrode in the array;
(c) incubating the microelectrode array carrying the immobilized substrates with the sample of interest and a nucleotide triphosphate comprising an electroactive-labelled gamma phosphate; and
(d) detecting the phosphorylation level of the substrates in each electrode, wherein phosphorylation of one or more substrates in the plurality of electrodes indicates the presence in the sample of the kinase specific to the one or more phosphorylated substrates.

In another aspect of the invention, a method of diagnosing in a subject a disease associated with abnormal levels or absence of a protein kinase is provided comprising:
(a) immobilizing a substrate of the kinase associated with the disease to one or more electrodes;
(b) incubating the one or more electrodes carrying the immobilized substrate with a sample from the subject and a nucleotide triphosphate comprising an electroactive-labelled gamma phosphate; and
(c) detecting the phosphorylation level of the substrates in the electrodes of each array, wherein an abnormal level or absence of phosphorylation in the subject's sample with respect to a normal control indicates that the subject has, or is susceptible to, the disease.

In a further aspect, there is provided a kinase biosensor comprising at least one kinase substrate immobilized on an electrode surface, wherein said electrode surface is immersed in an electrolyte comprising an electroactive nucleotide triphosphate having an electroactive-labelled gamma phosphate.

In yet another aspect, there is provided a kit for screening kinase phosphorylation characterised in that the kit comprises at least one kinase substrate, an electrode, the nucleotide triphosphate conjugate comprising an electroactive labelled gamma phosphate group and a kinase.

One or more advantages of at least some of these aspects include (i) novel electroactive nucleotide triphosphate conjugate suitable for monitoring and/or detecting events associated with phosphorylation, including phosphorylation itself, (ii) novel electroactive nucleotide triphosphate conjugate can be produced at a significantly lower cost compared to other methods of monitoring and/or defecting phosphorylation, (iii) methods of detecting or monitoring events associated with phosphorylation, including phosphorylation itself, do not require modification of peptides with an electro-active or optical label, and (iii) novel electroactive nucleotide triphosphate conjugate facilitates; simplifies and speeds the procedures involved with the monitoring and detection of phosphorylation events, including phosphorylation itself. In particular, the novel electroactive nucleotide triphosphate conjugate of the invention can be used in the discovery of new drugs, molecular diagnostics and molecular targeting.

These and other aspects of the invention will become apparent from the detailed description that follows, and the following figures in which:

### BRIEF DESCRIPTION OF THE DRAWINGS

FIG. 1 is a schematic illustrating the synthesis of an electroactive ferrocene-ATP conjugate;

FIG. 2 is a schematic illustrating the use of a metallocene-ATP conjugate in a method of electrochemically detect phosphorylation of a substrate;

FIG. 3 illustrates cyclic voltammograms obtained using various ferrocene-ATP concentrations (a-d) in the method of FIG. 2;

FIG. 4 illustrates cyclic voltammograms obtained in the presence (a) and absence (b) of PKC in the method of FIG. 2;

FIG. 5 illustrates square-wave voltammograms obtained in the presence (a) and absence (b) of PKC in the method of FIG. 2;

FIG. 6 graphically illustrates the dependence of current density responses on the reaction time of the method of FIG. 2;

FIG. 7 illustrates a microelectrode array; and

FIG. 8 (A-D) illustrates the use of a microelectrode array.

FIG. 9 (A) illustrates square-wave voltammograms of the CK2-catalyzed phosphorylation reactions performed in cell lysates containing (a) over-expressed CK2α, (b) endogenous CK2 levels, (c) the over-expressed kinase-dead CK2α, (d) normal-expressed CK2α.

FIG. 9 (B) is a plot for the detection of the CK2α over-expression state in Hela cell lysates.

FIG. 10 (A) is a cyclic voltammograms for CK2α′-catalyzed phosphorylation of substrate peptide in the presence (a)-(e) of different CK2α′ concentrations and in the absence (f) of the enzyme;

FIG. 10 (B) illustrates the effect of the CK2α′ concentration on the current responses using substrate peptide modified electrodes (a) in the assay buffer, (b) in the presence of HeLa cell lysate and (c) control experiment.

FIG. 11 (A) illustrates cyclic voltammograms for the inhibition of CK2α-catalyzed phosphorylation of the substrate peptide in the presence of the inhibitor, (1) TBB (4,5,6,7-Tetrabromo-2-azabenzimidazole) at different concentrations (a)-(e), and control (f) experiment in the absence of CK2α.

FIG. 11 (B) illustrates Lineweaver-Burk plot for the determination of kinetics of the CK2α′-catalyzed phosphorylation.

FIG. 11 (C) illustrates control experiments for FIG. 11 (A).

FIG. 12 (A) illustrates CV for the inhibition of tyrosine kinase-catalyzed phosphorylation with the Signal Transduction Protein (STP) peptide in the presence of (a)-(d) and in the absence (e) of Abl1-T315I.

FIG. 12 (B) Illustrates the Lineweaver-Burk plot for the determination of kinetics of the Abl1-T315I-catalyzed phosphorylation of the immobilized STP peptide

FIG. 12 (C) illustrates a plot for the dependence of the anodic current responses on the amount of the Abl1,T315I kinase in the presence of HeLa cell lysate with the STP peptide (a), (b) and control experiments (c).

FIG. 12 (D) illustrates a plot for the dependence of current responses on the concentration of the general protein kinase inhibitors (b) and (c) and control experiments (a).

FIG. 13(A) illustrates cyclic voltammograms for the inhibition of tyrosine kinase-catalyzed phosphorylation with the FLT3 peptide in the presence of HER2/ErbB2 at different concentrations (a), (b) and (c).

FIG. 13 (B) illustrates Lineweaver-Burk plot for the determination of kinetics of the HER2/ErbB2-catalyzed phosphorylation.

FIG. 13 (C) illustrates a plot for the dependence of the anodic current responses on the amount of the HER2/ErbB2 kinase in the presence (a) and absence (b) and (c) of the substrate peptide.

FIG. 13 (D) illustrates a plot for the dependence of J responses on the concentration of N-Benzoylstaurosporine.

FIG. 14 illustrates mass spectroscopy (MS) plot of kinase-catalized phosphorylation of substrate peptides.

### DETAILED DESCRIPTION OF THE INVENTION

A novel electroactive nucleotide triphosphate conjugate is provided. The nucleotide triphosphate comprises an electroactive-labelled gamma phosphate which is useful in a method of detecting phosphorylation activity of a kinase. In one embodiment, the method comprises immobilizing at least one substrate of the kinase on an electrode surface, incubating the immobilized substrate with the electroactive nucleotide triphosphate conjugate in the presence of the kinase under conditions which permit detection of phosphorylation activity and detecting phosphorylation of the substrate. In one aspect the phosphorylation activity is detected electrochemically. In another aspect the phosphorylation activity is detected by mass spectroscopy.

The term “electroactive” is used herein to denote that the transferable gamma phosphate comprises a label that is detectable on application of an electric field. Examples of an electroactive label include organic labels and organometallic labels. In one aspect the electroactive label includes a metallocene, including substituted metallocenes or a derivative thereof which is compatible with an aqueous environment. The metallocene may be, for example, ferrocene, cobaltocene or derivatives thereof. Substituted metallocenes such as halogen-substituted metallocenes, metallocene comprising an amide-substituted cyclopentadiene or other derivatives such as ansa-metallocenes, metallocenium cations such as ferrocenium, [Fe(C5H5)2]+, triple decker complexes (compounds with three Cp anions and two metal cations in alternating order, may also be used. In another aspect the electroactive label includes quinines, nitro heterocycles, NAD+, NADP+, nitrogen-containing aromatics and heterocycles.

The term “nucleotide triphosphate” is meant to refer to adenosine-5′-triphosphate (ATP) and nucleotide derivatives thereof, for example, comprising substituted adenosine derivatives at the 6 amino position. Substituents may include, for example, methoxy, ethoxy, pentyl, hexyl, benzyl and substituted benzyl as well as 5- and 6-membered ring structures comprising the nitrogen of the amino group.

In one embodiment, the electroactive nucleotide triphosphate may be a metallocene-ATP conjugate comprising a metallocene-labelled gamma phosphate formed by conjugation of a metallocene or derivative thereof to ATP. The metallocene-ATP conjugate may be formed using a synthetic protocol in which a carboxylated metallocene compound is treated to yield a Boc-protected or an N-protected conjugate that is combined with a reactive form of ATP to yield the desired conjugate. The identity of the metallocene-ATP conjugate may be confirmed using known techniques such as NMR spectroscopy or mass spectrometry to identify the phosphoramide bond at the γ position.

The electroactive nucleotide triphosphate, such as a metallocene-ATP conjugate, may be used in an assay to detect kinase-catalyzed phosphorylation. In one aspect of the present invention the assay includes an electrochemical assay, however other assays may be possible including mass spectroscopy. The conjugate is useful to detect the phosphorylation activity of any kinase including serine/threonine protein kinases such as PKC, KITR, PDFGR, CK2, CDKs, CDK2, MKK1, RAF, CHK1, mTOR, ROCK, MLK and P38/SAPK2a, as well as tyrosine kinases including receptor kinases such as EGRF, TRKA, TRKC, PDGFR-α and PDGFR-β, VEGFR1, VEGFR2, VEGFR3, ERBB2, ERBB3, ERBB4, MET, RON, EPHB2 and B4, RYK, DDR1, DDR2 and ALK and non-receptor tyrosine kinases such as SRC, SYK, ABL1, BRK, YES1 and JAK1-3.

Based on the target kinase, an appropriate substrate is selected for immobilization on a working electrode surface. Suitable working electrode surfaces include metals such as gold and platinum, semiconductor surfaces such as doped silicon or GaAs, and transparent conducting surfaces such as graphite, glassy carbon and indium tin oxide. The electrode surface, or working electrode may take the form of a micron size metal wire which is modified at the tip, or a chip-based electrode array in which each working electrode is individually addressable.

The electrode surface is coated with a kinase peptide substrate. In this regard, the peptide substrate may be modified at a terminal end thereof to include an entity that will bond to the electrode surface. The nature of the modification may vary with the nature of the electrode surface. For example, the substrate may be modified to include a terminal cysteine residue in order to permit attachment of the substrate to a metal electrode surface such as gold or Pt via an Au—S linkage or Pt—S linkage, respectively. For an ITO electrode surface, modification of the substrate to include a carboxylate residue is appropriate. For electrode surfaces comprising silicon, aminoalkyltriethoxysilane chemistry and peptide coupling strategies may be utilized. Coupling to carbon surfaces (glassy carbon and graphite) involve diazonium coupling of a benzoic acid derivative followed by peptide coupling of the kinase substrate peptide to the surface.

Examples of kinase substrates include, but are not limited to, AKTide-SA, AKTide-2T, Src Substrate II, CDK1 Substrate II, CdkS Substrate, Crebtide, Crosstide, Abl1 Signal Transduction Protein, HER2/ErbB2 FLT3 substrate, Syntide 2, Autocamtide-2, Autocamtide-3 and CK2 Substrate. Kinase substrates may comprise single or multiple phosphorylation sites. Multiple phosphorylation sites may be any one of tyrosine, serine or threonine.

The substrate-coated electrode surface is incubated with an electroactive nucleotide triphosphate such as a metallocene-ATP conjugate and a kinase of the substrate under conditions which permit detection of phosphorylation activity, such as, for example, electrochemically by immersion of the electrode surface in an electrolyte and the presence of a counter electrode such as a platinum wire, and a reference electrode such as Ag/AgCl or other reference electrode systems, such as calomel electrode, NHE (normal hydrogen electrode) and SHE (standard hydrogen electrode).

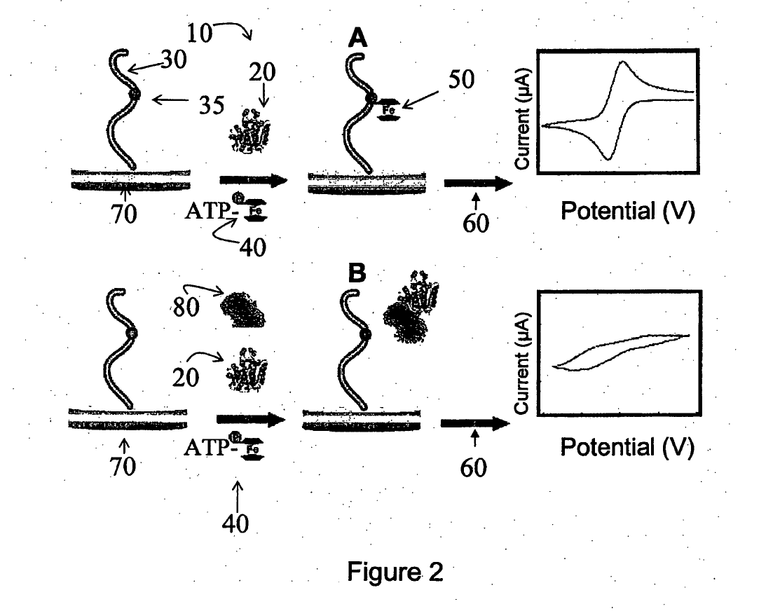

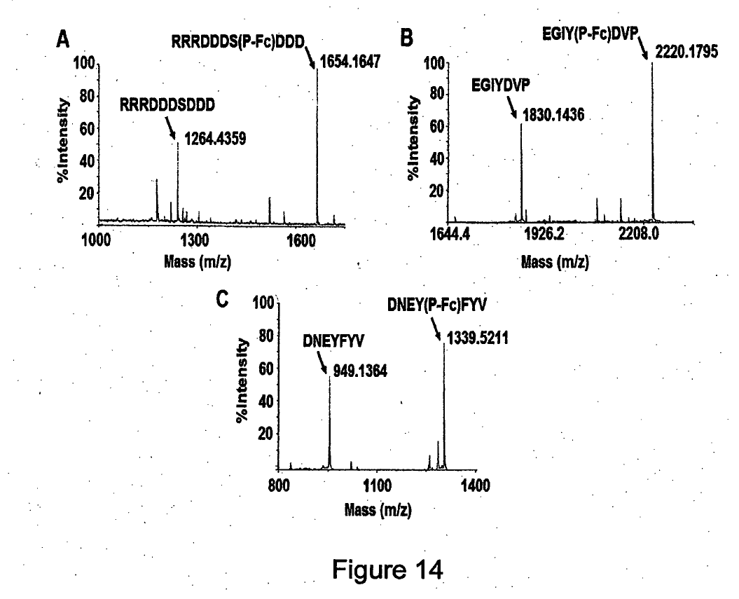

A schematic illustrating the reaction 10 that occurs on incubation is provided in FIG. 2(A) and illustrates that the kinase 20 delivers the electroactive gamma phosphate 50 of the metallocene-nucleotide triphosphate conjugate 40 to the substrate 30. Following incubation, phosphorylation of the substrate 30 with the electroactive gamma phosphate 50 of the nucleotide triphosphate 40 is detected 60 using a suitable electrochemical technique such as cyclic voltammetry, square-wave voltammetry and electrochemical impedance spectroscopy to measure the voltametric change or using other suitable techniques such as mass spectroscopy (see FIG. 14).

The methods of the present invention provide a means to identify the presence of a kinase in a solution such as a cell lysate, as well as a means to profile the activity of a kinase. The phosphorylation reaction is stoichiometric in that the voltametric change is directly proportional to the extent of phosphorylation measured by the transfer of the electroactive label such as a metallocene. Thus, the resulting electrode surface charge following phosphorylation is directly related to the total surface concentration of metallocene groups, thereby providing a quantitative means for measuring and determining phosphorylation rates in a rapid and precise fashion and allowing the monitoring of phosphorylation reactions in real time for kinase profiling. The reaction is also advantageously reversible, thereby allowing multiple uses of the substrate-modified electrode.

In addition, the methods of the present invention may be conducted in the presence of candidate kinase modulating compounds, including either inhibitor compounds or agonist compounds, providing a method of screening such candidates for their potential as therapeutic agents in connection with disease associated with a given kinase. Such a screening method, as illustrated in FIG. 2(B), comprises the steps of immobilizing a substrate 30 of a selected kinase 20 on an electrode surface 70, incubating the immobilized substrate 30 with an electroactive nucleotide triphosphate 40 such as a metallocene-ATP conjugate in the presence of the kinase 20 and a candidate compound 80 (such as an inhibitor) and detecting the level of phosphorylation of the substrate 30 by any suitable detection means 60 such as electrochemically or by mass spectroscopy. A change in the level of phosphorylation from the phosphorylation level that occurs in the absence of the candidate compound indicates that the candidate compound 80 modulates the activity of the kinase.

In addition, the methods of the present invention may be conducted to identify new protein kinase substrates. Such method comprises the steps of immobilizing a candidate substrate on an electrode surface, incubating the immobilized candidate with an electroactive nucleotide triphosphate such as a metallocene-ATP conjugate in the presence of a kinase and detecting the level of phosphorylation of the substrate by any suitable detection means such as electrochemically. Phosphorylation of the candidate substrate indicates that the candidate substrate is a substrate of the kinase.

In an embodiment of the invention, a microelectrode array is provided. The array comprises a series of electrodes to which are linked different peptide substrates, each of which is specific for a different protein kinase. The array is prepared similar to a single peptide substrate electrode with the exception that it includes multiple electrodes with varying substrates, and may comprise replicates of each substrate in order to yield statistically meaningful results. Each peptide substrate is modified at one of the C- or N-terminus to include a linking agent suitable to link it to the electrode surface as previously described. It will be appreciated by one of skill in the art that the kinases to be targeted by such an electrode array are not particularly restricted, and thus, the electrode array may comprise any selected peptide substrates.

A microelectrode array as described is useful for kinase profiling, including the determination of phosphorylation characteristics, of one or more kinases. In this regard, it is particularly useful to profile a cell lysate comprising a mix of components, and thus, is useful as a diagnostic tool to identify abnormal activity in a cell lysate in comparison to a standard, e.g. normal profile obtained from a healthy individual. The array is also useful to screen for kinase modulators to determine their effect on multiple kinase/substrate interactions in a single screen.

Abnormal protein phosphorylation is a cause of major diseases, including cancer, diabetes and chronic inflammatory diseases. For example, protein kinases CK2, Abl1 and HER2 are frequently over-expressed in tumours or leukemic cells and exhibit oncogenic activity in mice. Analytical methods to quantify protein kinase activity are critical for understanding their role in the diagnosis and therapy of these diseases. Accordingly, another aspect of the present invention is a method of diagnosing in a subject a disease associated with abnormal levels or absence of a protein kinase. Such method comprises the steps of immobilizing a substrate of the kinase associated with the disease to one or more electrodes; incubating the one or more electrodes carrying the immobilized substrate with a sample from the subject and a nucleotide triphosphate comprising an electroactive-labelled gamma phosphate; and detecting the phosphorylation level of the substrates in the one or more electrodes by any suitable detection means such as electrochemically, wherein an abnormal level or absence of phosphorylation in the subject's sample with respect to a normal control indicates that the subject has, or is susceptible to, the disease.

Subjects include any organism that has protein kinase in its system, including animals and plants.

Embodiments of the invention are described by reference to the following specific examples which are not to be construed as limiting.

### Example 1

### Synthesis of Fc-ATP

Preparation of Boc-NH(CH₂)₆N(H)COFc (Compound 1): Ferrocenecarboxylic acid (230 mg, 1 mmol) was dissolved in 20 mL anhydrous DCM. Then, 1.2 equiv. TEA (0.17 mL) and 1.2 equiv. HBTU (455 mg) were added sequentially. After 30 min., Boc-NH(CH₂)₆NH₂ was added to the solution and stirring was continued overnight. After reaction was completed, the solvent was removed in vacuo, and the residue was purified by flash column chromatography on silica gel (DCM-MeOH, 95:5; R_{f}=0.25) giving the desired compound as a yellow solid in 78% yield (334 mg). ¹H-NMR (δ, DMSO): 7.74 (t, 1H, J=5.2 Hz, NH—COFc), 6.78 (t, 1H, J=5.4 Hz, NH-Boc), 4.78 (s, 2H, Cp), 4.32 (s, 2H, Cp), 4.14 (s, 5H, Cp), 3.15 (q, 2H, J=6.4 Hz, CH₂), 2.90 (q, 2H, J=6.4 Hz, CH₂), 1.23-1.52 (m, 17H). ¹³C{¹H}-NMR (δ, DMSO): 168.57, 155.57, 77.27, 76.94, 69.73, 69.23, 68.06, 39.76, 38.54, 29.50, 29.47, 28.26, 26.17, 26.08. IR: νₘₐₓ=3363 (NH), 3310 (NH), 2976 (Fc), 2934 (Fc), 2861 (Fc), 1687 (CO-OtBu), 1623 (Amide-1), 1535 (Amide-2). MS (EI⁺) m/z: calc. for C₂₂H₃₂FeN₂O₃: 428.2. found: (M⁺) 428.1

Preparation of NH₂(CH₂)₆N(H)COFc (Compound 2): TFA (5 equiv.) was added to a mixture of Boc-protected ferrocenyl amine (334 mg, 1 mmol) in 10 mL DCM. After stirring the mixture for 1 h, the solvent was removed in vacuo. Three portions of DCM were added and evaporated to get rid of the excess TFA. The residue was dissolved in 10 mL DCM and 0.25 mL TEA was added to convert the TFA salt to free amine completely. After solvent removal, the mixture (contains TEAH+ salt) was used in the next step without further purification. For the purpose of characterization, the mixture was dissolved in 20 mL DCM (contains 5% TEA) and extracted with brine and water. After the removal of the solvent, the residue was dried in high vacuo to give a yellow solid. 90% yield (295 mg). ¹H-NMR (δ, DMSO-d₆): 7.74 (t, 1H, J=5.3 Hz, NH—COFc), 4.78 (s, 2H, Cp), 4.32 (s, 2H, Cp), 4.14 (s, 5H, Cp), 3.15 (q, 2H, J=6.4 Hz, CH₂), 2.52 (s, 2H, CH₂), 1.49 (t, 1H, J=6.6 Hz, CH2), 1.27-1.39 (m, 6H, CH2). ¹³C{¹H}-NMR (δ, DMSO): 168.56, 76.95, 69.70, 69.21, 68.05, 41.53, 38.58, 33.23, 29.51, 26.41, 26.24. IR: νₘₐₓ=3293.68 (NH₂), 2962.94 (Fc), 2928.63 (Fc), 2854.23 (Fc), 1624.45 (amide-1), 1541.51 (amide-2). MS (E1) m/z: calc. for C₁₇H₂₄FeN2O: 328.1. found: (M⁺) 328.1.

Preparation of γ-phosphate Fc-ATP (Compound 3): Adenosine 5′-triphosphate disodium salt (100 mg, 0.18 mmol) was dissolved in 10 mL 0.1 M TEAB buffer (pH=7.5) and loaded on a column packed with cation-exchange resin (AG 50W-X8), which has been pre-equilibrated with 0.1M TEAB buffer. The desired fraction (monitored by UV light) was collected and evaporated in vacuo. The residue was co-evaporated with 10 mL dry methanol three times and dissolved in 1.8 mL dry DMF under Argon. DCC (123 mg) was added and the mixture was stirred under Ar for 3 h at room temperature to form adenosine-5′-trimetaphosphate (ATMP). ATMP solution was added to a mixture of compound 2 (295 mg, 5 equiv.) in 10 mL MeOH and 0.25 mL TEA under Ar. The mixture was stirred for 30 min. and poured into 20 mL H₂O. The solution was loaded on a DEAE-cellulose column and washed with distilled H₂O to remove excess ferrocene-amine. Then, linear gradient of TEAB buffer (0.1-1 M) was carried out to give the desired fraction (yellow band), which was lyophilized into light yellow power. 50% yield of Fc-ATP (TEAH+ salt) and further exchanged TEAR to Na form for the NMR spectra. ³¹P {¹H}-NMR (δ, D₂O): −0.07 (γ) d, J=21.1 Hz; −10.76 (α) d, J=19.9 Hz; −22.14 (β) t, J=19.9 Hz. ¹H-NMR (δ, D₂O): 8.52 (s, 1H, H-8), 8.19 (s, 1H, H-2), 6.10 (d, 1H, J=5.5 Hz, H-1′), 4.73 (s, 2H, Cp), 4.74 (s, 1H, H-2′), 4.53 (s, 1H, H-3′), 4.46 (s, 2H, Cp), 4.37 (s, 1H, H-4′), 4.23 (m, 2H, H-5′), 4.20 (s, 5H, Cp), 3.16 (t, 2H, J=6.5 Hz, CH₂), 2.79 (q, 2H, J=7.8 Hz, CH₂), 1.31-1.45 (m, 4H, CH₂), 1.11-1.25 (m, 4H, CH₂). [H₄.M](ESI⁺) m/z: calc. for C₂₇H₃₉FeN₇O₁₃P₃: 818.1. found: 818.2.

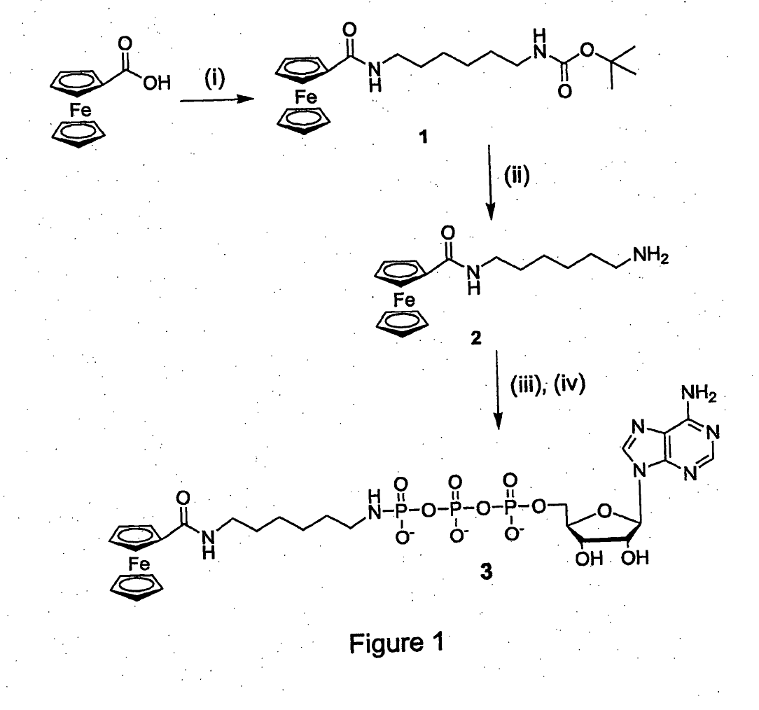

A schematic of the Fc-ATP conjugate synthesis is provided in FIG. 1.

Reagents: All synthesis reactions were carried out under an atmosphere of argon unless indicated otherwise. Diethylaminoethyl (DEAE)-cellulose, adenosine 5′-triphosphate (ATP) disodium salt was obtained from Sigma and used as received. Dowex AG 50W-X8 was obtained from Bio-Rad Laboratories (Ontario, Canada). N,N′-Dicyclohexylcarbodiimide (DCC), 0-(1H-Benzotriazol-1-yl)-N,N,N′,N′-tetramethyluronium hexafluorophosphate (HBTU) was obtained from AdvancedChemTech (KY, USA). Dimethylformamide (DMF) and dichloromethane (DCM) was distilled from CaH2 before use. Methanol was distilled from magnesium tuning with the presence of iodine. Ferrocenecarboxylic acid and t-butyl-6-aminohexylcarbamate⁸ were prepared according to the literature procedures.

### Example 2

### Electrochemical Detection of Protein Kinase C Phosphorylation

Cyclic voltammetry (CV) was performed using a CHInstruments 660 system (Austin, Tex.). DEP-chips with screen-printed gold electrodes (SPEs) were kindly donated by BioDevice Technology Ltd. (Ishikawa, Japan) and prepared as set out in Li et al. Anal. Chem. 2005, 77 5766-5769. The total length of an SPE was 11 mm, and the geometric area of the working electrode was 2.64 mm². The reference electrode was a Ag/AgCl past electrode and the counter electrode was a carbon electrode.

¹H, ¹³C, ³¹P NMR experiments were performed on a Bruker Avance 500 MHz spectrometer and chemical shifts were referenced to the residue DMSO (2.50 ppm for ¹H and 39.52 ppm for ¹³C) and H₂O (4.79 ppm). Mass spectrometry was carried out using a Perkin Elmer-Sciex API 365 instrument.

Unless otherwise specified, reagents were purchased from Merck. All solutions were prepared and diluted using ultra-pure water (18.3 MΩ-cm) from the Millipore Milli Q system.

### 1. Protein Kinase C-Catalyzed Phosphorylation Reaction Using Fc-ATP

The SPEs were incubated in petri-dishes at room temperature throughout the preparatory steps in order to avoid rapid evaporation of the solutions on the surfaces. The electrochemical measurements were performed three times for each condition (n=3), except as otherwise stated.

### 2. Immobilization of the Protein Kinase C Substrate Peptides on SPEs

An aliquot of 200 μM substrate protein kinase CC peptide solution (5 μL) was allowed to coat the gold working electrode of the SPEs and was incubated overnight at 4° C. The protein kinase Cζ peptide (SIYRRGSRRWRKL) was purchased from Calbiochem (EMD Biosciences, USA) and modified with a cysteine residue at the N-terminus. The modified protein kinase CC pseudosubstrate sequence contains Ser119 instead of Ala119.⁹

After the incubation step, the electrodes were washed with blank TBS. The peptide film was diluted by immersing the SPEs in 0.1 mM ethanolic solution of hexanethiol for 5 min and rinsing the surface with blank TBS.

### 3. PKC-Catalyzed Phosphorylation on the SPE Surface

Kinase assay buffer included 20 mM Tris, 0.5 mM EDTA, 10 mM MgCl₂, 500 μg/mL phosphatidyl serine (pH 7.5). The concentrations of Fc-ATP and kinase, PKC, were varied according to the optimum experimental conditions. Protein kinase C from rat brain (E. C. 2.7.1.37) was purchased from Sigma in 50% glycerol containing 20 mM Tris, 0.5 mM EDTA, 0.5 mM EGTA, 5 mM DTT, 100 mM NaCl, 0.02% Tween 20, and 1 μg/mL leupeptin. One unit (U) of PKC will transfer 1 nanomole of phosphate from ATP into histone H1 per min at 30° C.^{10,11} The aliquots (200 μL) of the optimized assay buffer including 100 U/mL PKC and 100 μM Fc-ATP were added into 1.5-mL vials.

Substrate peptide-immobilized SPEs were placed in the vials incubated at 30° C. for 1 h in a heating block (VWR Scientific, USA). After 1 h of incubation, the SPEs were washed with blank TBS to remove the excess Fc-ATP and other reagents, and then placed in the electrochemical workstation.

### 4. Electrochemical Measurement on SPCE Surface

Electrochemical detection was performed by spotting 20 μL of 0.1 M NaClO₄ (pH 6.5) onto the surface of SPE at room temperature. Cyclic voltammetry (CV) was performed at a scan rate of 100 mV/s. Square-wave voltammetry (SWV) involved the oxidation of Fc residues by sweeping the potential from 0 to 1 V with an amplitude of 25 mV at 15 Hz frequency.

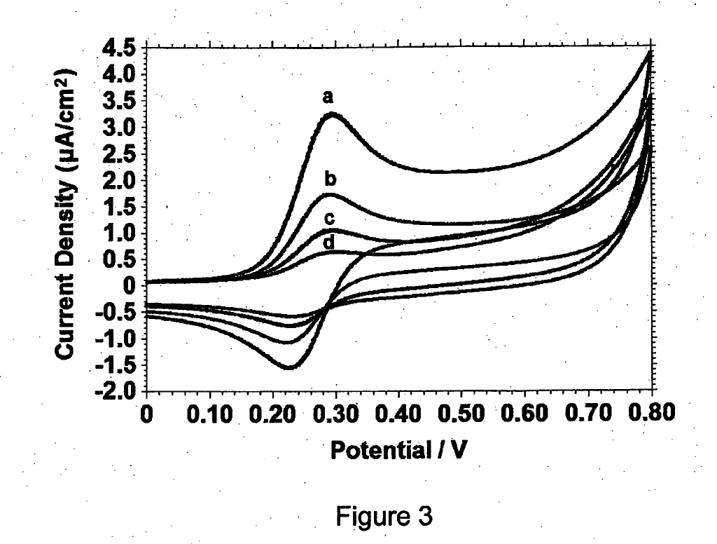

Schematic illustration of the electrochemical principal for the detection of kinase-catalyzed phosphorylation using Fc-ATP as the co-substrate is shown in FIG. 2(A). The substrate peptide 30 is immobilized on the surface of the SPE 70 via a sulphur bond. Protein kinase 20 C (PKC)-catalyzed reaction transfers γ-phosphate-Fc group 50 to the serine 35 residue of the peptide 30. The Fc group 50 attached to the peptide 30 is electrochemically observed using CV. The voltammetric detection of Fc involves the scanning of the potential range between 0 and 1 V at a rate of 100 mV/s. As a result of this electrochemical process the reversible redox properties of Fc-ATP were monitored. FIG. 3 shows the voltammetric responses obtained from the CV of Fc-ATP in solution in the presence of (a) 100 μM (b) 50 μM (c) 25 μM and (d) 10 μM Fc-ATP in solution.

The oxidation peak was detected at ˜0.26 V and the reduction peak was observed at ˜0.22 V (vs. Ag paste-based reference electrode of the SPE). The separation of the redox peak potentials indicated that one electron was involved in the process. This electrochemical behaviour was expected from the well-defined electrochemical properties of Fc.

For the optimization of experimental conditions, a series of measurements were taken in the presence of varying Fc-ATP concentrations and 100 U/mL PKC using the same assay conditions. As the concentration of Fc-ATP increased, the phosphorylation of the peptides resulted in the high current responses on the surface. The current responses remained the same for concentrations over 100 μM. Thus, 100 μM Fc-ATP was applied for further kinase assays. When no ATP-F was used in the assay buffer, no significant current response was obtained indicating the suppression of non-specific adsorption of Fc-ATP on the electrode surface by the stringent washing of the SPEs as described. When low concentrations of Fc-ATP were used, no current responses were observed.

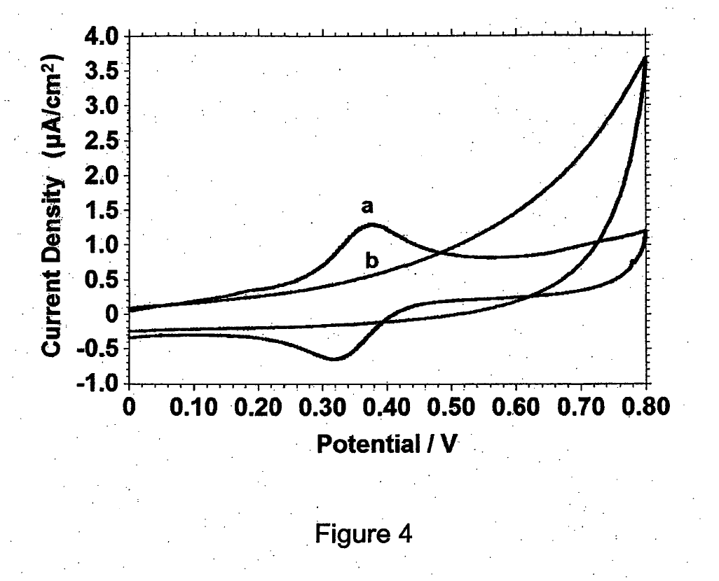

Using the surface-immobilized peptides, the current density responses were recorded in the presence and absence of PKC in the assay solution as shown in FIG. 4. The CV response shown in FIG. 4-a shows the similar redox behaviour of Fc-ATP as observed in solution, however, the peak potentials were slightly shifted to higher values indicating the presence of a peptide film on the surface, which hampered the redox process to occur at a lower potential. The absence of any redox current signals in FIG. 4-b indicated that the attachment of Fc-ATP to the peptides was dependent on the presence of the kinase. Moreover, no redox activity in the absence of PKC showed the successful suppression of the non-specific adsorption of Fc-ATP on the electrode surface.

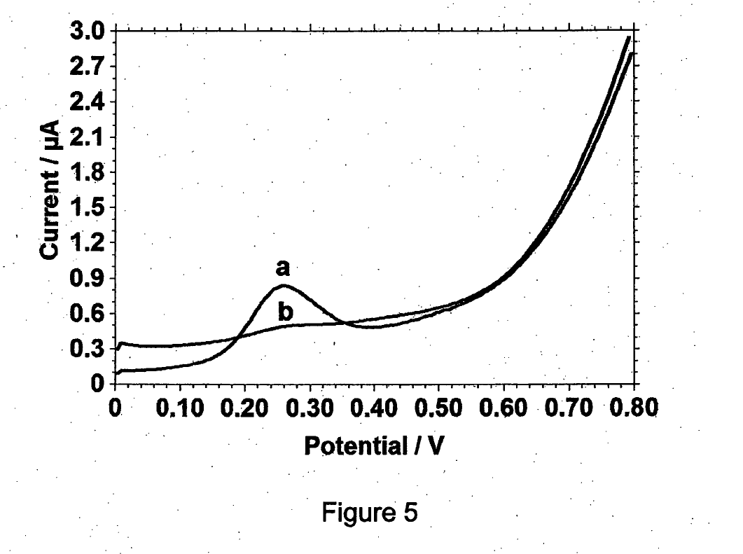

SWV was also applied to detect the Fc oxidation current signals at low concentrations of PKC as shown in FIG. 5. The substrate peptide and Fc-ATP concentration was kept constant at 200 μM and 100 μM, respectively. FIG. 5-a shows the current response obtained in the presence of 0.1 U/mL PKC while FIG. 5-b shows the current response obtained in the presence of 0.01 U/mL PKC. The increasing trend of the current density responses were recorded, as the concentration of PKC increased (FIG. 5).

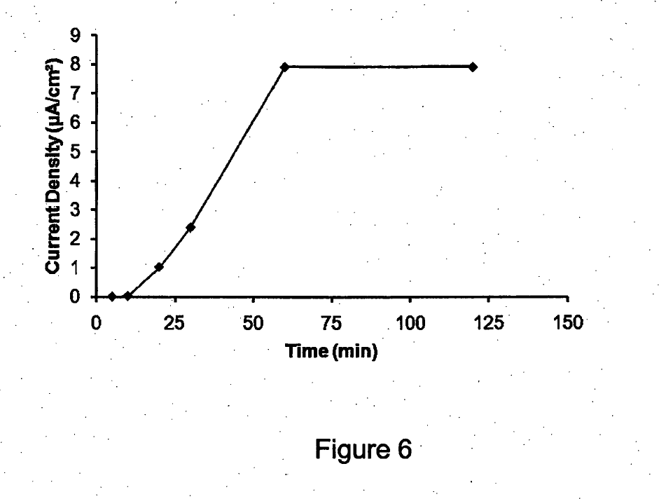

The dependence of incubation time was monitored for the optimization of Fc-ATP responses. The concentrations of substrate peptide, PKC and Fc-ATP were kept constant at 200 μM, 100 U/mL and 100 μM, respectively, and the dependence of the current responses on incubation time at 30° C. was recorded as shown in FIG. 6. The peak current heights reached a saturation level, when the assay solution was incubated for 1 h. When the kinase reaction was allowed to continue only for 20 min, a small current response was observed indicating that the surface-immobilized substrate peptides were not phosphorylated efficiently in the presence of 100 μM Fc-ATP (FIG. 6).

### Example 3

### Detection of Casein Kinase 2 (CK2) and Tyrosine Kinases Abl1 and HER2/ErbB2 Phosphorylation

It was previously demonstrated that using the nucleotide triphosphate conjugate comprising an electroactive labelled gamma phosphate group is useful to detect protein kinase C activity using an electrochemical biosensing system. In this example the utility of the nucleotide triphosphate conjugate was used to detect another well-described protein serine/threonine kinase, casein kinase-2 (CK2) and two clinically important tyrosine kinases, Abl1 and HER2/ErbB2 and to evaluate this method for measuring protein kinase inhibitor potency.

First the enzymatic modification of kinase-specific peptide RRRDDDSDDD¹² for the serine/threonine kinase, CK2 was evaluated using mass spectroscopy with Fc-ATP as the co-substrate.

FIG. 14 shows MS plot for the kinase-catalyzed phosphorylation of the substrate peptides for (A) CK2, (B) Abl1-T315I and (C) HER2/ErbB2 using Applied Biosystems 4700 Proteomics Analyzer with DHB (2,5-Dihydrobenzoic acid) matrix (10 mg/mL) and 1:1 mixture with the sample. The sample containing the phosphorylated peptides were enriched and purified using the standard protocol of Phosphopeptide Isolation kit (ThermoScientific Pierce).

Our results (FIG. 14A) clearly demonstrate that CK2 transfers the desired redox group to the target peptide (m/z CK2 target peptide before: 1264.4359, after Fc transfer: 1654.1647). Additional reactions were carried out using the substrate peptides for Abl1-T315I and HER2/ErbB2, clearly showing the utility of our approach also for tyrosine kinases (FIGS. 14B & C).

### 1. Materials and Methods

i. Immobilization of the Substrate Peptides on SPEs

The covalent immobilization of substrate peptides on the gold microelectrode surface using succinimide-esters of lipoic acid included the following steps: (a) incubation of the bare gold microelectrode with 5 mM NHS-lipoic acid ester in ethanol for 15 h; (b) incubation of the N-Hydroxysuccinimide (NHS)-lipoic acid-modified surface with the substrate peptides in the presence of 2 mM N-(3-Dimethylaminopropyl)-N′-ethylcarbodiimide (EDC) in 0.1 M 2-(N-Morpholino) ethanesulfonic acid (MES, pH 6) for 2 h, (c) incubation of the peptide-modified electrodes with Mercaptopolyethylene glycol 5′000 monomethyl ether (PEG-thiol 5′000) solution (1:100 v/v) in ethanol for 10 min.

ii. CK2-Catalized Phosphorylation

CK2α and CK2α′ kinases assay buffer included 50 mM Tris HCl (pH 7.5), 10 mM MgCl₂, 150 mM NaCl. The concentration of ATP-Fc was 100 μM ATP-Fc in a total reaction volume of 25 μL. CK2α and CK2α′ forms of CK2 and the peptide substrate (RRRDDDSDDD) were prepared in D. W. Litchfield's laboratory (University of Western Ontario, London, Canada). The substrate peptide modified electrodes were incubated at 37° C. for 2 h. After the incubation period, the electrodes were washed multiple times using 2 M NaClO₄. After the washing process, the electrodes were immersed into 2 M NaClO₄ for the electrochemical measurement using Ag/AgCl reference electrode, which was connected with the electrolyte via a salt bridge and a Pt wire was used as the counter electrode.

iii. Abl1-T315I-Catalyzed Phosphorylation

Abl1-T315I kinase assay buffer included 60 mM HEPES (pH 7.5), 5 mM MgCl₂, 5 mM MnCl₂, 3 μM Na₃VO₄, 400 μM ATP, peptide substrate (STP) and the kinase in a total reaction volume of 25 μL. Purified recombinant human Abl1-T315I mutant kinase and its substrate peptide Signal Transduction Protein (STP, EGIYDVP) were purchased from Cell Signalling Technology (MA, USA). The substrate peptide modified electrodes were incubated at 37° C. for 2 h. After following the same washing procedures as for CK2, the CV measurements were recorded using the same parameters as described above.

iv. HER/ErbB2 Catalysed Phosphorylation

The activity of HER2/ErbB2 kinase was measured using the following conditions: 5 mM MOPS (pH 7.2), 2.5 mM (3-glycerophosphate, 5 mM MnCl₂, 100 μM Fc-ATP in the presence of FLT3 peptide substrate and 10 ng/μL kinase in a total reaction volume of 25 μL. Purified recombinant human HER2/ErbB2 kinase and FLT3 (DNEYFYV) substrate peptide were purchased from Cell Signalling Technology (MA, USA). The substrate peptide modified electrodes were incubated at 37° C. for 2 h. After following the same washing procedures as described above for CK2, the CV measurements were recorded using the same parameters.

v. Cell-Lysate Pre-Treatment

For the reactions containing cell lysates, 20×106 HeLa cells that were obtained from D. W. Litchfield's laboratory (University of Western Ontario, London, Canada), were lysed in 1 mL of lysis buffer (50 mM Tris (pH 8), 150 mM NaCl, 10% Glycerol, 0.5% Triton X-100™) containing 1 mM phenylmethanesulphonylfluoride (PMSF, Pierce, USA) by rotation for 10 minutes. During the phosphorylation reactions, Halt phosphatase inhibitor cocktail (Pierce, USA) in 1:1 ratio (v/v) with the cell lysate was used for suppressing the serine, threonine and tyrosine phosphatase activities. The cell debris was collected at 12,000 rpm and the supernatant was stored at 4° C. until use in subsequent reactions. For the reactions containing HeLa lysates, the lysate solution was mixed with the kinase reaction buffer at a ratio of 1:10 (v/v) and applied to the phosphorylation or dephosphorylation reactions as described above.

Vi. Calculation of Enzymatic Activity Using Electrochemical Data

The electrochemical data from the CV measurements are obtained as charge density (J) per test. The concentration of Fc-ATP for the kinase activity determinations was 100 μM, for CK2 and 200 μM for Abl1-T315I and HER2-ErbB2. Here, the detailed description of the calculation procedure will be given for CK2-catalysed phosphorylation reactions. The reaction volume is 25 μL, which leads to 2.5 nmole Fc-ATP per test. Then, the specific electro-activity (SE) of Fc-ATP (J/nmole Fc-ATP) would be calculated as follows:

$$SE ( J \text{/} nmole ) = \frac{{J}_{total}}{[ Fc \text{-} ATP ]} \qquad ( 1 )$$

Then, the specific activity of CK2 is calculated with the following formula:

$$Activity ( µmole \cdot {min}^{- 1} \cdot {µL}^{- 1} ) = \frac{( Δ J \times Dil \times 25 )}{( SE \times Vol \times T )} \qquad ( 2 )$$

where ΔJ represents (J sample-J blank) and Dil is the dilution factor with a total reaction volume of 20 μL with time (T) in minutes of reaction and the enzyme volume (V) in μL.

The apparent inhibition constants Ki′ were determined by fitting equation (3) to the experimental data.

$$V = \frac{{V}_{0}}{1 + [ I ] / {K}_{i}^{′}} \qquad ( 3 )$$

where V is the rate, V0 is the rate in the absence of the inhibitor, [I] is the inhibitor concentration and Ki′ is the apparent inhibition constant. The true inhibition constants Ki were calculated by correction of Ki′ according to Equation (4):

$${K}_{i} = \frac{{K}_{i}^{′}}{1 + [ S ] / {K}_{m}} \qquad ( 4 )$$

where [S] is the surface density of the immobilized substrate peptide and Km is the Michaelis-Menten constant. The surface density conditions of the immobilized substrate peptide were changed between 1, 5, 10, 15 and 20 pmol/cm2. The initial time dependence of the kinase reactions were determined at these varying peptide density conditions. The phosphorylation reaction was stopped after 5, 15, 30, 60, 90, 120 and 150 min, and the measurement of the attached Fc molecules was carried out. The reciprocals of these current values were plotted against the reciprocal of the peptide density, which gave linear Lineweaver-Burk curve. The equation of this curve defines the kinetic data, where the y intercept is 1/Vmax. If the y is set for 0 and the equation is solved for x, x intercept becomes equal to −1/Km.

### 2. Results

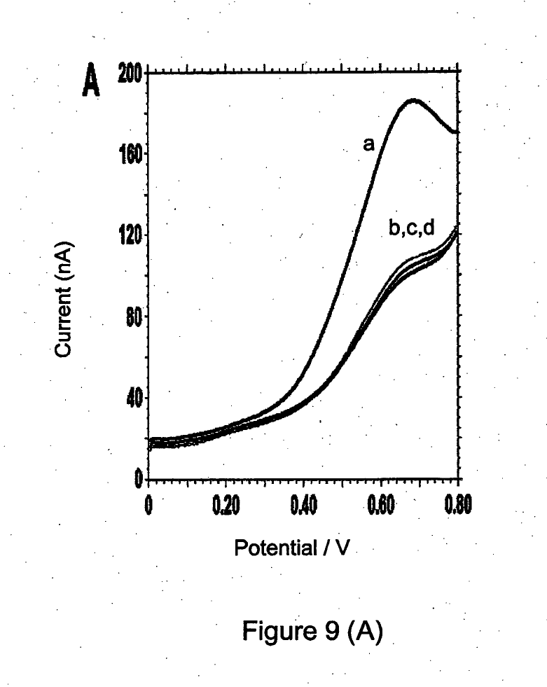

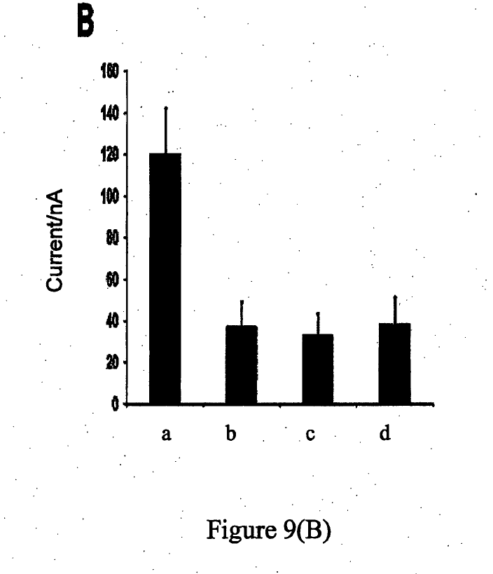

FIG. 9 (A) illustrates the square wave voltammetry of the CK2-catalysed phosphorylation reactions performed in the cell lysates. The substrate peptide modified gold microelectrodes were immersed into the cell lysates containing (a) the over-expressed CK2α, (b) endogenous CK2 levels, (c) the over-expressed kinase-dead CK2α, (d) normal-expressed CK2α. The measurements were taken as described above; (B) Plot for the detection of the CK2α over-expression state in cell lysates. The increase in the current signal indicates that the kinase was in excess amount and could cause the attachment of a larger amount of Fc molecules on the peptides in comparison with the other cell lysates.

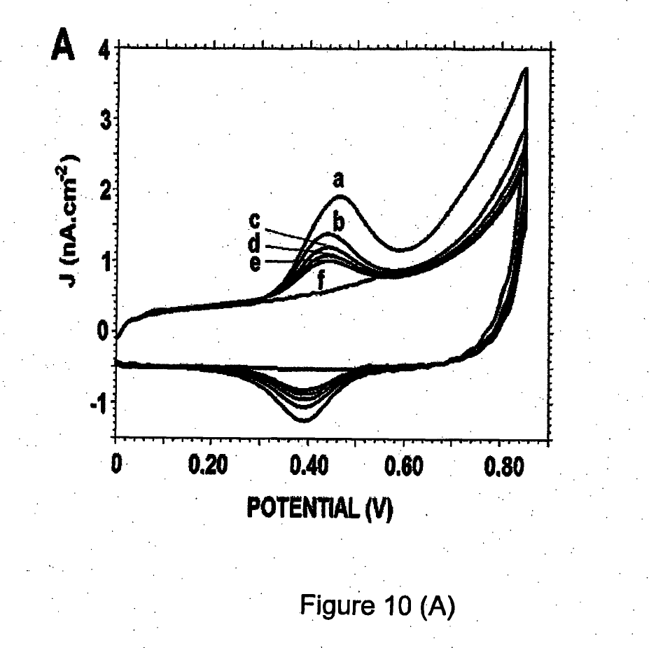

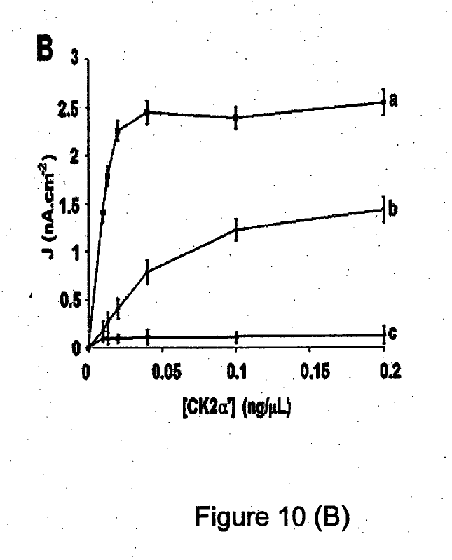

FIG. 10 shows: (A) Cyclic voltammograms (CV) for CK2α′-catalyzed phosphorylation of substrate peptide (RRRDDDSDDD) in the presence of CK2α′ at a concentration of (a) 0.04, (b) 0.02, (c) 0.008, (d) 0.005, (e) 0.0025 ng/μL, and (f) control experiment in the absence of the enzyme; (B) Effect of the CK2α′ concentration on the current responses using substrate peptide modified electrodes (a) in the assay buffer, (b) in the presence of HeLa cell lysate, and (c) control experiment was performed using the substrate peptide for Abl1-T315I (EGIYDVP) in the presence of cell lysate.

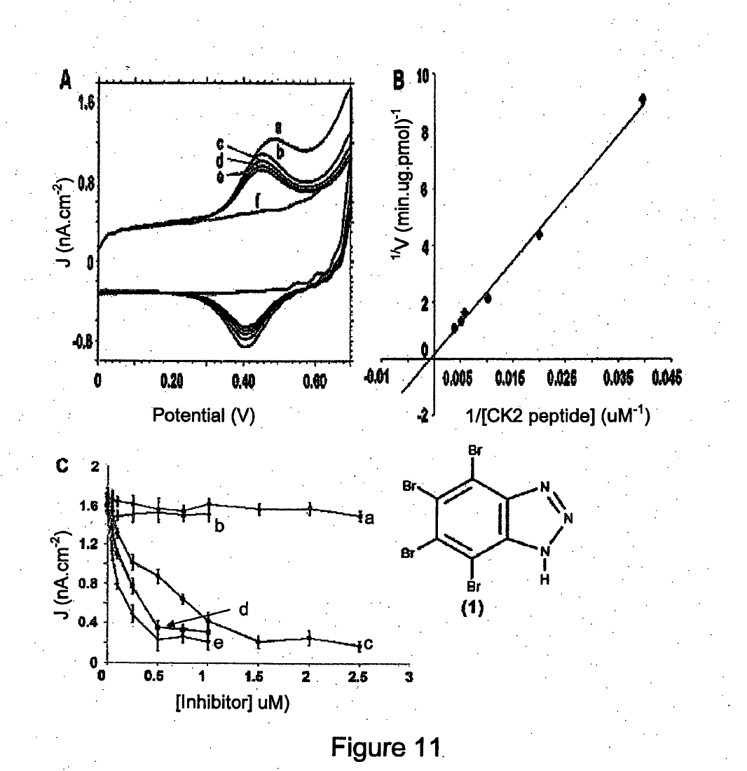

FIG. 11 shows: (A) cyclic voltammograms for the inhibition of CK2α-catalyzed phosphorylation with the substrate peptide in the presence of the inhibitor, (1) TBB (4,5,6,7-Tetrabromo-2-azabenzimidazole) (a) 250 nM, (b) 500 nM, (c) 750 nM, and (d) 800 nM (e) 900 nM, and (f) control experiment in the absence of CK2α; (B) Lineweaver-Burk plot for the determination of kinetics of the CK2α′-catalyzed phosphorylation of the immobilized substrate peptide at varying surface density conditions on the Au microelectrode surface as described in the text; (C) Control experiments were performed by (a) titrating the phosphorylated substrate peptide on the surface with the assay buffer, which showed the stability of the electrochemical responses, and (b) EGIYDVP was not inhibited upon exposure to (1) and demonstrated the specificity of the inhibition reactions, (c) the current responses decreased rapidly in the presence of (2-Dimethylamino-4,5,6,7-tetrabromo-1H-benzimidazole) and (d) (E-3-(2,3,4,5-Tetrabromophenyl)acrylic acid) in the HeLa cell lysate.

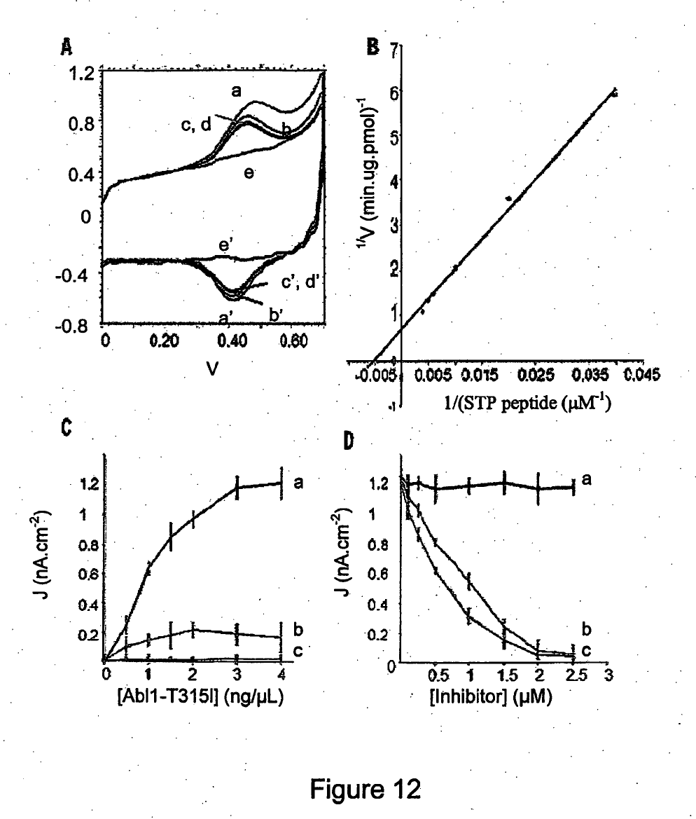

FIG. 12 shows: (A) CV for the inhibition of tyrosine kinase-catalyzed phosphorylation with the Signal Transduction Protein (STP) peptide (EGIYDVP at a surface density 12 pmol·cm−2) in the presence of Abl1-T315I at a concentration of (a) 3, (b) 1.5, (c) 1, (d) 0.75 ng/μL, (e) in the absence of the enzyme, no current responses were observed, which indicated the suppression of non-specific adsorption; (B) Lineweaver-Burk plot for the determination of kinetics of the Abl1-T315I-catalyzed phosphorylation of the immobilized STP peptide at varying surface density conditions on the Au microelectrode surface as described in the text; (C) Plot for the dependence of the anodic current responses on the amount of the Abl1-T315I kinase in the presence of HeLa cell lysate with (a) the STP peptide immobilized on the surface, (b) a control experiment using the FLT3 peptide (DNEYFYV), which a preferable substrate for HER2/ErbB2 at a surface density 12.5 pmol·cm−2. Low current responses indicated insufficient phosphorylation between the FLT3 peptide and the Abl1-T3151, (c) a second control experiment involved the CK2 substrate peptide (RRRDDDSDDD) at a surface density 10 μmol·cm⁻². No significant current responses were observed, which evidenced the specificity of the phosphorylation reaction; (D) Plot for the dependence of current responses on the concentration of the general protein kinase inhibitors, (b) Staurosporine and (c) N-Benzoylstaurosporine, the control experiments involved the titration of the phosphorylated STP peptide with the buffer titration in the presence of HeLa cell lysates (a). No significant drops were observed in the current responses indicating that the contents of the cell lysate did not affect the current signals.

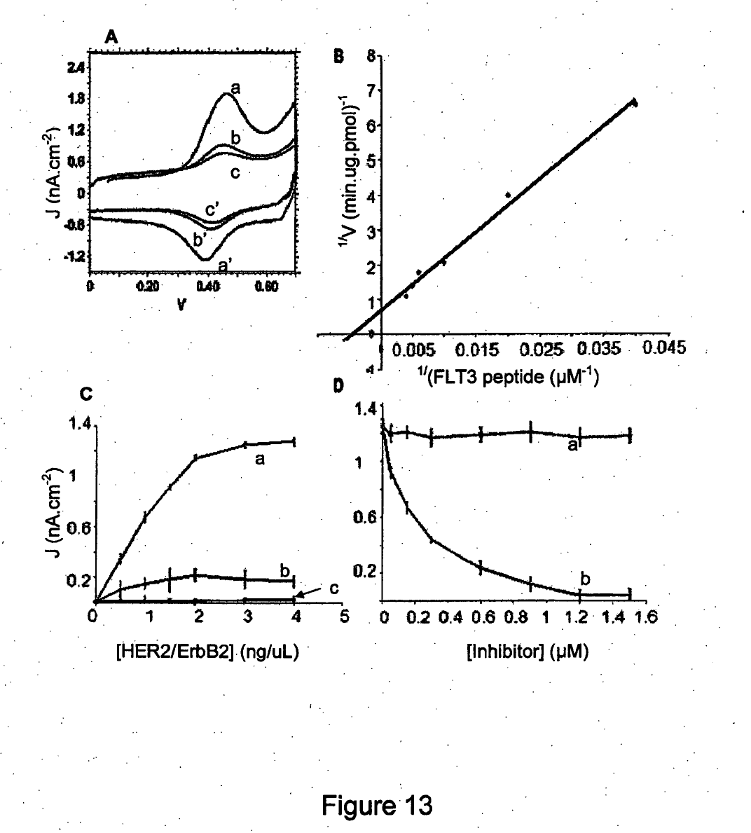

FIG. 13 shows: (A) Cyclic voltammograms for the inhibition of tyrosine kinase-catalyzed phosphorylation with the FLT3 peptide (DNEYFYV at a surface density 12.5 pmol·cm−2) in the presence of HER2/ErbB2 at a concentration of (a) 4, (b) 0.5, (c) 0.25 ng/μL; (B) Lineweaver-Burk plot for the determination of kinetics of the HER2/ErbB2-catalyzed phosphorylation of the immobilized FLT3 peptide at varying surface density conditions on the electrode surface; (C) Plot for the dependence of the anodic current responses on the amount of the HER2/ErbB2 kinase with (a) the FLT3 peptide immobilized on the surface in the presence of HeLa cell lysate, (b) control experiment with STP peptide (EGIYDVP) resulted in a slight increase in the current responses, (c) the CK2 substrate peptide (RRRDDDSDDD) at a surface density 10 pmol·cm−2 in the presence of HeLa cell lysate; (D) Plot for the dependence of J responses on the concentration of (b) N-Benzoylstaurosporine, whereas (a) the phosphorylation of the immobilized FLT3 peptide was not affected with blank buffer titration in the presence of HeLa cell lysates.

i. Measuring Kinase Activity in Cellular Extracts

The application of a solution containing recombinant CK2 and Fc-ATP into Hela cell lysates resulted in robust phosphorylation of the immobilized peptide substrate as indicated by cyclic voltammetry measurements of the surface-confined Fc molecules. As shown in FIG. 9A, the highest intensity of the square-wave voltammetry (SWV) current signal was observed for the lysates derived from cells with elevated CK2 levels. By comparison, lower current responses were obtained from the other cell lysates including uninduced cells or cells expressing kinase-inactive CK2α. Overall, the kinase activity measurements obtained by electrochemical detection are in complete correspondence with measurements previously obtained using conventional radioactive detection of CK2 activity. In the assays performed with these lysates, the reduction signal of the oxidized Fc+ was not observed (or shifted outside the scanned potential window) possibly due to the presence of numerous proteins inhibiting the reduction. FIG. 9B displays the average SWV current responses obtained from a set of five measurements performed on the same peptide in the cell lysate environment under the same conditions.

ii. Phorphorylation Specificity

The phosphorylation of the immobilized peptides using the nucleotide triphosphate conjugate of the present invention is specific. Protein tyrosine kinases (Abl-T315I and HER2) did not catalyze phosphorylation of the CK2 peptide substrate (FIGS. 12C-c and 13C-c), and CK2 did not catalyze phosphorylation a peptide that was the preferred substrate of Abl-T315I (EGIYDVP, FIG. 10B-c).

iii. Modulators of Kinase Activity

Based on the demonstration of the electrochemical technique of the present invention for the detection of the reversible phosphorylation of a kinase substrate, this method was adapted to evaluate small molecule inhibitors acting on these enzymes. Therefore, the ability of the peptide biosensor to assess the inhibitory activity of three recently-developed CK2 inhibitors was evaluated (FIG. 11). Reactions were performed with CK2, Fc-ATP, and each inhibitor (at concentrations ranging from 50 nM to 2.5 μM) were exposed onto the CK2 substrate peptide films. After incubation for 2 h at 37° C. with CK2α, the biosensors were washed and analysed by electrochemical measurements. Table 1 shows the inhibition data for the analysis Ki values for four kinases and five inhibitors in total. In general, the Ki values that were calculated from the electrochemical data are in agreement with literature values obtained with conventional kinase assays.^{13-17.}

TABLE 1
Comparison of kinetic constants of protein kinases with their substrate
peptides immobilized on gold microelectrodes. The kinetic data were
extracted from measurements using varying surface density conditions
of the substrate peptides immobilized on the surface.

| | | | Vmax | |
|---|---|---|---|---|
| | | | (μmol · | Vmax/ |
| Protein Kinase | Peptide | Km (mM) | min-1 · mg-1) | Kmax |
| CK2α | RRRDDDSDDD | 0.087 | 1.97 | 22.64 |
| CK2α′ | RRRDDDSDDD | 0.098 | 1.78 | 18.16 |
| Abl1-T315I | EGIYDVP | 0.182 | 1.67 | 9.18 |
| HER2/ErbB2 | DNEYFYV | 0.208 | 1.54 | 7.41 |

To evaluate the utility of the electrochemical biosensor for the measurement of other kinase assays, the biosensors were modified to monitor Abl1 and HER2 protein tyrosine kinases: The first FDA-approved kinase inhibitor drug, Imatinib (Gleevec™) has been successfully used to treat Bcr-Ab1 kinase associated chronic myeloid leukemia.¹⁸ The most frequently identified mutation associated with resistance to Gleevec™ is T315I in the Abl1 kinase domain.^{19,20,21} Another successful small molecule inhibitor is Trastuzumab (Herceptin®), which is used as part of a treatment regimen containing doxorubicin, cyclophosphamide, and paclitaxel for the adjuvant treatment of patients with HER2-overexpressing, node-positive breast cancer.^{22,23} Specific activities for the two CK2 isoforms, Abl1 and HER2 on their substrate peptides are shown in Table 2. Notably, the activities determined by the electrochemical measurements compare very favourably with the literature values^{1(a)}. However, the slightly low reaction rates seen in the electrochemical assays may arise in part through decreased accessibility of the substrate peptides anchored on the Au electrode surface.

TABLE 2
Comparison of Ki of small molecule inhibitors on protein kinases with
their substrate peptides immobilized on gold microelectrodes. The Ki
values were determined with the data obtained using varying surface
density conditions of the substrate peptides immobilized on the surface.

| Inhibitor (nM) | CK2α | CK2α′ | Abl1-T315I | HER2/ErbB2 |
|---|---|---|---|---|
| (1) | 450 | 380 | — | — |
| (2) | 35 | 20 | — | — |
| (3) | 50 | 25 | — | — |
| Staurosporine | — | — | 550 | 225 |
| N-Benzoylstaurosprine | — | — | 600 | 275 |

Protein tyrosine kinases were also challenged with the well-defined general inhibitors of kinases, staurosporine and its derivative, N-benzoylstaurosporine (FIGS. 12D and 13D), which are ATP-competitive inhibitors with broad-spectrum inhibitory activities. Again, the Ki values for Staurosporine and its derivative that were determined by electrochemical assays were very similar to those obtained using conventional radioactive assays for Abl1^{19,20,21} and HER2.²⁴

### Example 4

### Preparation of a Microelectrode Array

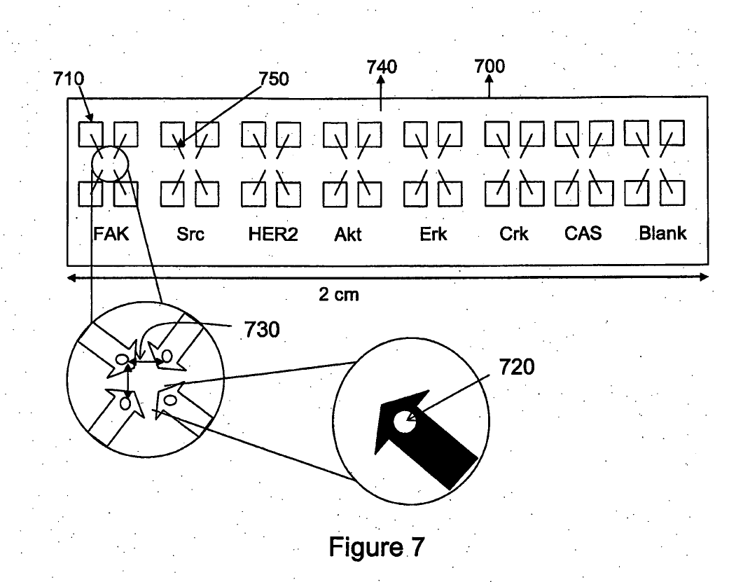

A microelectrode array 700 for use in the present method to determine the phosphorylation characteristics of multiple protein kinases is shown in FIG. 7. The protocol for its fabrication is the same as that described in Li et al. 2005; however, a new lithographic mask is prepared based on the design shown in FIG. 7. An 800-nm silicon dioxide insulating layer is thermally grown on a p-type silicon wafer. A gold layer. (200 nm) is deposited onto a titanium adhesion layer (20 nm) sputtered onto the Si chip. Both metal layers are photolithographically patterned using Shipley 1813 photoresist 740 as a mask layer using the mask of FIG. 7. Etching of the metal layers is achieved as described previously (Li et al. Anal. Chem. 2006, 78, 6096-6101).²⁵ The individual gold micropads 720 possess a 10 micrometer diameter and are separated from each other by a distance of 50 micrometers 730. Each micropad 720 will be addressable from an external pad 710 through a microwire (1 mm²) 750. The arrangement of the micropads 720 into quadruples facilitates the spotting of individual peptide substrates.

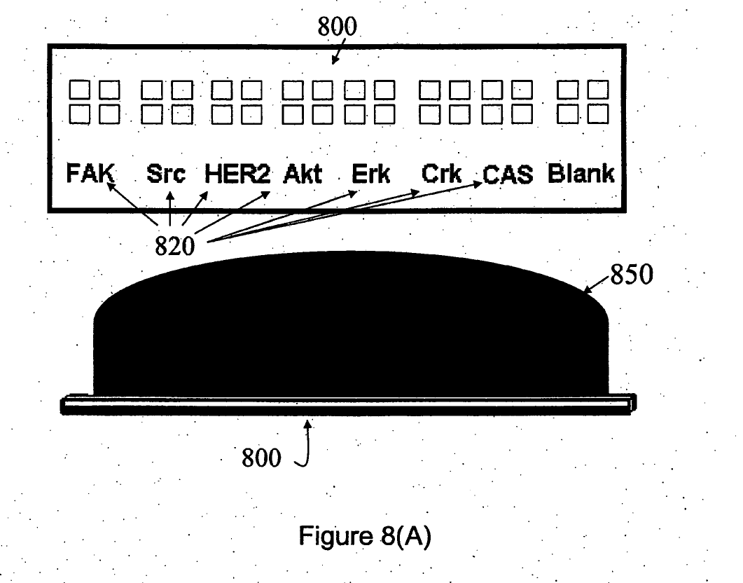

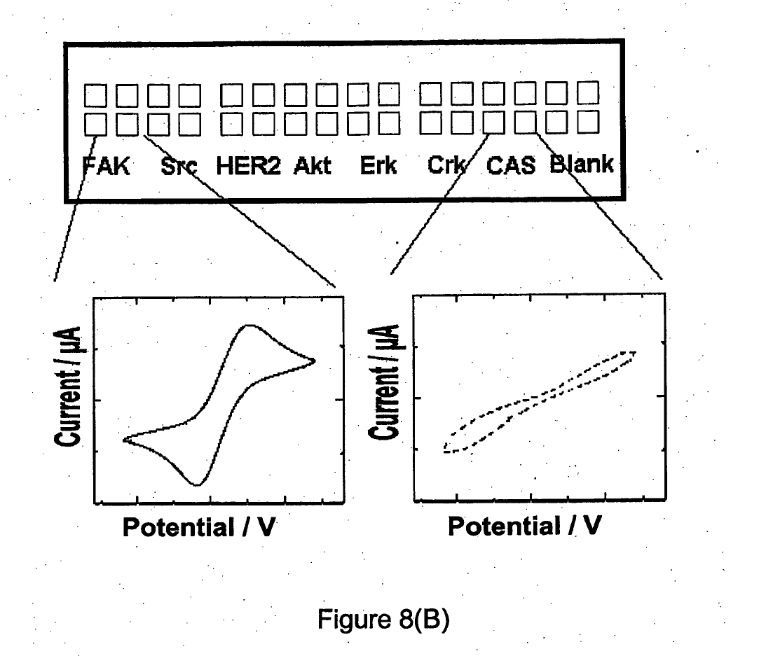

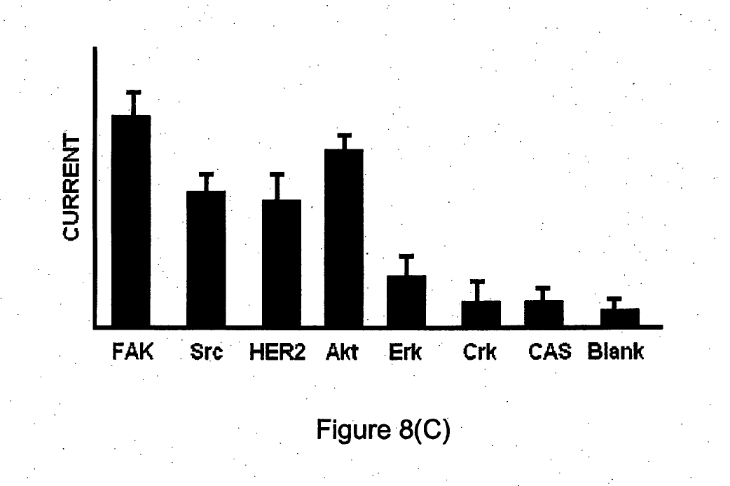

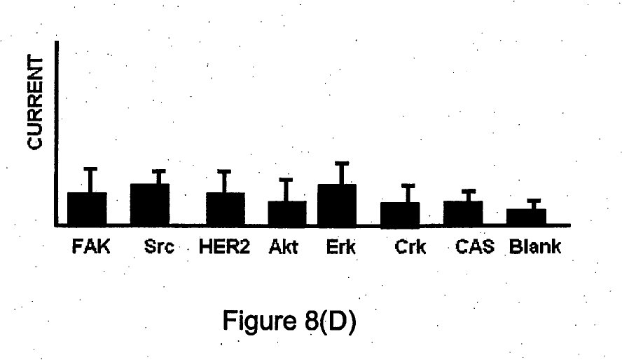

An example of the use of a microelectrode array 800 is illustrated in FIG. 8.

A kinase substrate peptide related to different kinases 820 are immobilized on the microelectrode array 800 (A). In this example, the kinase related to breast cancer are immobilized on the chip 800: FAK, Src, HER2, Akt, Erk, Crk, CAS. Substrates will be incubated with cell lysates 850 and the phosphorylation reaction will take place in the presence of ferrocene (Fc)⁻ conjugated ATP. After the phosphorylation reaction, electrochemical measurements will be performed at each microelectrode (B). FIGS. 8(C) and 8(D) illustrate the average square-wave voltammetry current responses obtained with the set of kinases 820 and a blank for individuals with breast cancer (FIG. 8(D)) and healthy individuals (FIG. 8(E)). The statistical evaluation of the data for breast cancer will not only help the diagnosis of cancer or other diseases states, but also the effect of the small molecule inhibitors on the phosphorylation process can be determined. The difference of the electrochemical responses between the samples obtained from healthy and cancer individuals will provide rapid diagnosis and therapeutic follow-up possibilities.

### REFERENCES

1. (a) P. Cohen, Nat. Cell Biol. 2002, 4, E127-E130; (b) G. Burnett and E. P. Kennedy, J. Biol. Chem. 1954, 211, 969-980; (c) E. H. Fischer and E. G. Krebs, J. Biol. Chem. 1955, 216, 121-132 (d) E. H. Fischer, D. J. Graves, E. R. S. Crittenden and E. G. Krebs, J. Biol. Chem. 1959, 234, 1698-1704.
2. I. Melnikova and J. Golden, Nat. Rev. Drug Discov. 2004, 3, 993-994; (b) J. S. Sebolt-Leopold and R. Herrera, Nat. Rev. Cancer 2004, 4, 937-947; (c) J. M. Yingling, K. L. Blanchard and J. S. Sawyer, Nat. Rev. Drug Discov. 2004, 3, 1011-1022; (d) M. J. A. de Jonge and J. Verweij, Eur. J. Cancer 2006, 42, 1351-1356; (e) P. Cohen, Nat. Rev. Drug Discov. 2002, 1, 309-315; (f) G. Manning, D. B. Whyte, R. Martinez, T. Hunter and S. Sudarsanam, Science 2002, 298, 1912-1934.
3. (a) B. E. Turk, J. E. Hutti and L. C. Cantley, Nat. Protoc., 2006, 1, 375-379; (b) C. J: Hastie, H. J. McLauchlan and P. Cohen, Nat. Protoc., 2006, 1, 968-971; (c) B. T. Houseman, J. H. Huh, S. J. Kron and M. Mrkisch, Nat. Biotechnol., 2002, 20, 270-274; (d) A. F. Braunwalder, D. R. Yarwood, T. Hall, M. Missbach, K. E. Lipson and M. A. Sills, Anal. Biochem., 1996, 234, 23-26; (e) W. Tegge, R. Frank, F. Hoffmann and W. R. Dostmann, Biochemistry, 1995, 87, 99-106.
4. (a) M. Sato, T. Ozawa, K. Inukai, T. Asano and Y. Umezawa, Nat. Biotechnol., 2002, 20, 287-294; (b) M. Sato, T. Ozawa, T. Yoshida and Y. Umezawa, Anal. Chem. 1999, 71, 3948-3954; (c) M. Sato and Y. Umezawa, Methods, 2004, 32, 451-455; (d) Y. Umezawa, Biosens. Bioelectron. 2005, 20, 2504-2511; (e) K. Tomizaki and H. Mihara, Mol. BioSyst., 2006, 2, 580-589.
5. (a) M. D. Allen, L. M. DiPilato, M. Randar, Y. R. Ren, C. Chong, J. O. Liu and J. Zhang, ACS Chem. Biol., 2006, 1, 371-376; (b) D. M. Rothman, M. D. Shults and B. Imperiali, Trends Cell Biol., 2005, 15, 502-510; (c) M. D. Shults, K. A. Janes, D. A. Lauffenburger, B. Imperiali, Nat. Methods 2005, 2, 277-283; (d) M. D. Shults and B. Imperiali, J. Am. Chem. Soc. 2003, 125, 14248-14249.
6. (a) K. D. Green and M. K. H. Pflum, J. Am. Chem. Soc., 2007, 129, 10-11; (b) Z. Wang, R. Levy, D. G. Fernig and M. Brust, J. Am. Chem. Soc., 2006, 128, 2214-2215; (c) Z. Wang, J. Lee, A. R. Cossins and M. Brust, Anal. Chem., 2005, 77, 5770-5774.
7. A. Sonoda and I. J. Moritani, Organomet. Chem. 1971, 26, 133-140.
8. R. Leung-Toung, T. F. Tam, Y. Zhao, C. D. Simpson, W. Li, D. Desilets and K. J. Karimian, Org. Chem. 2005, 70, 6230-6241.
9. S. Osada, K. Mizuno, T. C. Saido, K. Suzuki, T. Kuroki and S. Ohno, Mol. Cell. Biol. 1992, 12, 3930-3938.
10. U. Kikkawa, A. Kishimoto and Y. Nishizuka, Ann. Rev. Biochem., 1989, 58, 31-41.
11. G. M. Walton, P. J. Bertics, L. G. Hudson, T. S. Vedvick and G. N. Gill, Anal. Biochem., 1987, 161, 425-437.
12. Kuenzel, E. A.; Mulligan, J. A.; Sommercorn, J.; Krebs, E. G. J. Biol. Chem. 1987, 262, 9136-9140.
13. Litchfield, D. W. Biochem. J. 2003, 369, 1-15.
14. Zien, P.; Duncan, J. S.; Skierski, J.; Bretner, M.; Litchfield, D. W.; Shugar, D. Biochim. Biophys. Acta 2005, 1754, 271-280.
15. Battistutta, R.; Mazzorana, M.; Sarno, S.; Kazimierczuk, Z.; Zanotti, G.; Pinna, L. A. Chem. Biol. 2005, 12, 1211-1219.
16. Sarno, S.; De Moliner, E.; Ruzzene, M.; Pagano, M A.; Battistutta, R.; Bain, J.; Fabbro, D.; Schoepfer, J.; Elliot, M.; Furet, P.; Meggio, F.; Zanotti, G.; Pinna, L. A. Biochem. J. 2003, 374, 639-646.
17. Pagano, M. A.; Meggio, F.; Ruzzene, M.; Andrzejewska, M.; Kazimierczuk, Z.; Pinna, L. A. Biochem. Biophys. Res. Commun. 2004, 321, 1040-1044.
18. Pagano, M. A.; Poletto, G.; Di Maria, G.; Cozza, G.; Ruzzene, M.; Sarno, S.; Bain, J.; Elliott, M.; Moro, S.; Zagotto, G.; Meggio, F.; Pinna, L. A. Chem Bio Chem 2007, 8, 129-139.
19. Capdeville, R.; Buchdunger, E.; Zimmerman, J.; Matter, A. Nat. Rev. Drug. Discov. 2002, 1, 493-502.
20. Sebolt-Leopold, J. S.; English, J. M. Nature 2006, 441, 457-462.
21. Weisberg, E.; Manley, P. W.; Cowan-Jacob, S. W.; Hochhaus, A.; Griffin, J. D. Nat. Rev. Cancer 2007, 7, 345-356.
22. Maekawa, T.; Ashihara, E.; Kimura, S.; Kimura, S. Int. J. Clin. Oncol. 2007, 12, 327-340.
23. Hicks, D. G.; Kulkami, S. Am. J. Clin. Pathol. 2008, 129, 263-273.
24. Dittadi, R.; Gion, M. J. Nat. Cancer Inst. 2000, 92, 1443-1444.
25. Tikhomirov, O.; Carpenter, G. J. Biol. Chem. 2001, 276, 33675-33680
26. X. Li, J. S. Lee, H.-B. Kraatz, Analytical Chemistry 2006, 78, 6096.

1. A nucleotide triphosphate conjugate comprising an electroactive labelled gamma phosphate group.

2. The nucleotide triphosphate conjugate of claim 1 characterised in that the electroactive labelled gamma phosphate group is selected from the group comprising an organic labelled gamma phosphate group and organometallic labelled gamma phosphate group.

3. The nucleotide triphosphate conjugate of claim 1 characterised in that the electroactive labelled gamma phosphate group is a metallocene, including substituted metallocenes.

4. The nucleotide triphosphate conjugate of claim 3 characterised in that the metallocene is selected from the group comprising of: ferrocene, cobaltocene and any derivatives thereof.

5. The nucleotide triphosphate conjugate of claim 1 characterised in that the electroactive labelled gamma phosphate group is selected from the group comprising of quinones and nitro heterocycles.

6. The nucleotide triphosphate conjugate of claim 1 characterised in that the nucleotide triphosphate comprises an adenosine-5′-triphosphate (ATP) and nucleotide derivatives thereof.

7. The nucleotide triphosphate conjugate of claim 6 characterised in that the nucleotide derivatives include substituted adenosine derivatives.

8. (canceled)

9. (canceled)

10. A method of detecting the phosphorylation of a substrate characterised in that the method comprises:
(a) immobilizing the substrate on an electrode surface;
(b) incubating the immobilized substrate with a kinase-containing electrolyte and a nucleotide triphosphate conjugate comprising an electroactive labelled gamma phosphate group; and
(c) detecting the phosphorylation of the substrate.

11. The method of claim 10 characterised in that the substrate includes single or multiple phosphorylation sites.

12. The method of claim 11 characterised in that the phosphorylation sites in the substrate include tyrosine, serine or threonine residues.

13. The method of claim 10 characterised in that the kinase comprises serine/threonine protein kinases and tyrosine kinases.

14. The method of claim 10 characterised in that the substrate is a candidate kinase substrate, wherein phosphorylation of the candidate substrate indicates that said candidate is a substrate of the kinase.

15. The method of claim 10 characterised in that the electroactive labelled gamma phosphate group is selected from the group comprising an organic and organometallic labelled gamma phosphate group.

16. The method of claim 10 characterised in that the electroactive labelled gamma phosphate group is a metallocene, including substituted metallocenes.

17. The method of claim 16 characterised in that the metallocene is selected from the group comprising of: ferrocene, cobaltocene and any derivatives thereof.

18. The method of claim 10 characterised in that the electroactive labelled gamma phosphate group is selected from the group comprising of: quinones and nitro heterocycles.

19. The method of claim 10 characterised in that the nucleotide triphosphate comprises an adenosine-5′-triphosphate (ATP) and nucleotide derivatives thereof.

20. (canceled)

21. The method of claim 10 characterised in that the electrode is selected from the group comprising of: a screen-printed gold electrode, a gold micro electrode, a gold microelectrode array chip, a carbon electrode and Indium tin oxide (ITO) electrodes.

22. The method of claim 10 characterised in that said phosphorylation is detected electrochemically.

23. (canceled)

24. A method of detecting a kinase of interest in a sample characterised in that the method comprises:
(a) immobilizing a substrate specific for the kinase of interest on an electrode surface;
(b) incubating the immobilized substrate with the sample and a nucleotide triphosphate conjugate comprising an electroactive labelled gamma phosphate group; and
(c) detecting phosphorylation of the substrate,
wherein phosphorylation of the substrate indicates the presence of the kinase in the sample.

25. (canceled)

26. (canceled)

27. (canceled)

28. (canceled)

29. The method of claim 24 characterised in that the electroactive labelled gamma phosphate group is selected from the group comprising an organic and organometallic labelled gamma phosphate group.

30. The method of claim 24 characterised in that the electroactive labelled gamma phosphate group is a metallocene, including substituted metallocenes.

31. The method of claim 30 characterised in that the metallocene is selected from the group comprising of: ferrocene, cobaltocene and any derivatives thereof.

32. The method of claim 24 characterised in that the electroactive labelled gamma phosphate group is an organic labelled gamma phosphate group, wherein said organic labelled group is selected from the group comprising of quinones and nitro heterocycles.

33. The method of claim 24 characterised in that the nucleotide triphosphate comprises an adenosine-5′-triphosphate (ATP) and nucleotide derivatives thereof.

34. (canceled)

35. The method of claim 24 characterised in that the electrode is selected from the group comprising of: a screen-printed gold electrode, a gold micro electrode, a gold microelectrode array chip, a carbon electrode and ITO electrodes.

36. (canceled)

37. The method of claim 24 characterised in that said phosphorylation is detected electrochemically.

38. (canceled)

39. A method of screening candidate compounds that modulate kinase activity characterised in that the method comprises:
(a) immobilizing a substrate of a kinase on an electrode surface;
(b) incubating the immobilized substrate with a kinase-containing electrolyte, the candidate compound and a nucleotide triphosphate comprising an electroactive-labelled gamma phosphate group; and
(c) detecting the phosphorylation level of the substrate, wherein a change in the phosphorylation level from a level of phosphorylation achieved in the absence of said compound indicates that said compound modulates the activity of the kinase.

40-44. (canceled)

45. The method of claim 39 characterised in that the electroactive labelled gamma phosphate group is selected from the group comprising an organic and organometallic labelled gamma phosphate group.

46. The method of claim 39 characterised in that the electroactive labelled gamma phosphate group is a metallocene, including substituted metallocenes.

47. The method of claim 46 characterised in that the metallocene is selected from the group comprising of: ferrocene, cobaltocene and any derivatives thereof.

48. The method of claim 39 characterised in that the electroactive labelled gamma phosphate group is an organic labelled gamma phosphate group, wherein said organic labelled group is selected from the group comprising of: quinones and nitro heterocycles.

49. The method of claim 39 characterised in that the nucleotide triphosphate comprises an adenosine-5′-triphosphate (ATP) and nucleotide derivatives thereof.

50. (canceled)

51. The method of claim 39 characterised in that the electrode is selected from the group comprising of: a screen-printed gold electrode, a gold micro electrode, a gold microelectrode array chip, a carbon electrode and ITO electrodes.

52. The method of claim 39 characterised in that said phosphorylation is detected electrochemically.

53. (canceled)

54. A method of high-throughput screening a sample for the presence of protein kinases characterised in that the method comprises:
(a) providing a microelectrode array comprising a plurality of electrodes;
(b) immobilizing kinase substrates to each electrode in the array;
(c) incubating the microelectrode array carrying the immobilized substrates with the sample of interest and a nucleotide triphosphate comprising an electroactive-labelled gamma phosphate group; and
(d) detecting the phosphorylation level of the substrates in each electrode, wherein phosphorylation of one or more substrates in the plurality of electrodes indicates the presence in the sample of the kinase specific to the one or more phosphorylated substrates.

55. (canceled)

56. (canceled)

57. The method of claim 54 characterised in that the sample is a fluid selected from the group consisting of: a cell culture, a cell lysate, an extract, a body fluid, and a purified protein solution.

58. (canceled)

59. A method of diagnosing in a subject a disease associated with abnormal levels or absence of a protein kinase, characterised in that the method comprises:
(a) immobilizing a substrate of the kinase associated with the disease to one or more electrodes;
(b) incubating the one or more electrodes carrying the immobilized substrate with a sample from the subject and a nucleotide triphosphate conjugate comprising an electroactive-labelled gamma phosphate group;
(c) detecting the phosphorylation level of the substrates in the electrodes of each array, wherein an abnormal level or absence of phosphorylation in the subject's sample with respect to a normal control indicates that the subject has, or is susceptible to, the disease.

60-63. (canceled)

64. A kinase biosensor characterised in that the biosensor comprises at least one kinase substrate immobilized on an electrode surface, wherein said electrode surface is immersed in an electrolyte comprising an electroactive nucleotide triphosphate having an electroactive-labelled gamma phosphate group.

65. A kit for screening kinase phosphorylation characterised in that the kit comprises at least one kinase substrate, an electrode, a nucleotide triphosphate conjugate comprising an electroactive labelled gamma phosphate group and a kinase.

66. The kit of claim 65 characterised in that the kinase substrate is immobilized to the electrode.

67. (canceled)

68. (canceled)


SEQUENCE LISTING (4 sequences)

SEQ ID NO: 1 - protein, 13 aa, Artificial Sequence
  Description of Artificial Sequence Synthetic peptide
SIYRRGSRRWRKL

SEQ ID NO: 2 - protein, 10 aa, Artificial Sequence
  Description of Artificial Sequence Synthetic peptide
RRRDDDSDDD

SEQ ID NO: 3 - protein, 7 aa, Artificial Sequence
  Description of Artificial Sequence Synthetic peptide
EGIYDVP

SEQ ID NO: 4 - protein, 7 aa, Artificial Sequence
  Description of Artificial Sequence Synthetic peptide
DNEYFYV